SEQUENCE LISTING (6 sequences)

SEQ ID NO: 1 - protein, 1706 aa, Bordetella pertussis
MQQSHQAGYANAADRESGIPAAVLDGIKAVAKEKNATLMFRLVNPHSTSLIAEGVATKGLGVHAKSSDWGLQAGYIPVNPNLSKLFGRAPEVIARADNDVNSSLAHGHTAVDLTLSKERLDYLRQAGLVTGMADGVVASNHAGYEQFEFRVKETSDGRYAVQYRRKGGDDFEAVKVIGNAAGIPLTADIDMFAIMPHLSNFRDSARSSVTSGDSVTDYLARTRRAASEATGGLDRERIDLLWKIARAGARSAVGTEARRQFRYDGDMNIGVITDFELEVRNALNRRAHAVGAQDVVQHGTEQNNPFPEADEKIFVVSATGESQMLTRGQLKEYIGQQRGEGYVFYENRAYGVAGKSLFDDGLGAAPGVPSGRSKFSPDVLETVPASPGLRRPSLGAVERQDSGYDSLDGVGSRSFSLGEVSDMAAVEAAELEMTRQVLHAGARQDDAEPGVSGASAHWGQRALQGAQAVAAAQRLVHAIALMTQFGRAGSTNTPQEAASLSAAVFGLGEASSAVAETVSGFFRGSSRWAGGFGVAGGAMALGGGIAAAVGAGMSLTDDAPAGQKAAAGAEIALQLTGGTVELASSIALALAAARGVTSGLQVAGASAGAAAGALAAALSPMEIYGLVQQSHYADQLDKLAQESSAYGYEGDALLAQLYRDKTAAEGAVAGVSAVLSTVGAAVSIAAAASVVGAPVAVVTSLLTGALNGILRGVQQPIIEKLANDYARKIDELGGPQAYFEKNLQARHEQLANSDGLRKMLADLQAGWNASSVIGVQTTEISKSALELAAITGNADNLKSVDVFVDRFVQGERVAGQPVVLDVAAGGIDIASRKGERPALTFITPLAAPGEEQRRRTKTGKSEFTTFVEIVGKQDRWRIRDGAADTTIDLAKVVSQLVDANGVLKHSIKLDVIGGDGDDVVLANASRIHYDGGAGTNTVSYAALGRQDSITVSADGERFNVRKQLNNANVYREGVATQTTAYGKRTENVQYRHVELARVGQLVEVDTLEHVQHIIGGAGNDSITGNAHDNFLAGGSGDDRLDGGAGNDTLVGGEGQNTVIGGAGDDVFLQDLGVWSNQLDGGAGVDTVKYNVHQPSEERLERMGDTGIHADLQKGTVEKWPALNLFSVDHVKNIENLHGSRLNDRIAGDDQDNELWGHDGNDTIRGRGGDDILRGGLGLDTLYGEDGNDIFLQDDETVSDDIDGGAGLDTVDYSAMIHPGRIVAPHEYGFGIEADLSREWVRKASALGVDYYDNVRNVENVIGTSMKDVLIGDAQANTLMGQGGDDTVRGGDGDDLLFGGDGNDMLYGDAGNDTLYGGLGDDTLEGGAGNDWFGQTQAREHDVLRGGDGVDTVDYSQTGAHAGIAAGRIGLGILADLGAGRVDKLGEAGSSAYDTVSGIENVVGTELADRITGDAQANVLRGAGGADVLAGGEGDDVLLGGDGDDQLSGDAGRDRLYGEAGDDWFFQDAANAGNLLDGGDGRDTVDFSGPGRGLDAGAKGVFLSLGKGFASLMDEPETSNVLRNIENAVGSARDDVLIGDAGANVLNGLAGNDVLSGGAGDDVLLGDEGSDLLSGDAGNDDLFGGQGDDTYLFGVGYGHDTIYESGGGHDTIRINAGADQLWFARQGNDLEIRILGTDDALTVHDWYRDADHRVEIIHAANQAVDQAGIEKLVEAMAQYPDPGAAAAAPPAARVPDTLMQSLAVNWR

SEQ ID NO: 2 - protein, 1706 aa, Bordetella bronchiseptica
MQQSHQAGYANAADRESGIPAAVLDGIKAVAKEKNATLMFRLVNPHSTSLIAEGVATKGLGVHAKSSDWGLQAGYIPVNPNLSKLFGRAPEVIARANNDVNSSLAHGHTAVDLTLSKERLDYLRQAGLVTGMADGVVASNHAGYEQFEFRVKETSDGRYAVQYRRKGGDDFEAVKVIGNAAGIPLTADIDMFAIMPHLSNFRDSARSSVTSGDSVTDYLARTRRAASEATGGLDPERIDLLWKIARAGARSAVGTEARRQFRYDGDMNIGVITDFELEVRNALNRRAHAVGAQDVVQHGTEQNNPFPEADEKIFVVSATGESQMLTRGQLKEYIGQQRGEGYVFYENRAYGVAGKSLFDDGLGAAPGVPGGRSKSSPDVLETVPASPGLRRPSLGAVERQDSGYDSLDGVGSRSFSLGEVSDMAAVEAAELEMTRQVLHAGARQDDAEPGVSGASAHWGQRALQGAQAVAAAQRLVHAIALMTQFGRAGSTNTPQEAASLSAAVFGLGEASSAVAETVSGFFRGSSRWAGGFGVAGGAMALGGGIAAAVGAGMSLTDDAPAGQKAAAGAEIALQLTGGTVELASSIALALAAARGVTSGLQVAGASAGAAAGALAAALSPMEIYGLVQQSHYADQLDKLAQESSAYGYEGDALLAQLYRDKTAAEGAVAGVSAVLSTVGAAVSIAAAASVVGAPVAVVTSLLTGALNGILRGVQQPIIEKLANDYARKIDELGGPQAYFEKNLQARHEQLANSDGLRKMLADLQAGWNASSVIGVQTTEISKSALELAAITGNADNLKSADVFVDRFIQGERVAGQPVVLDVAAGGIDIASRKGERPALTFITPLAAPGEEQRRRTKTGKSEFTTFVEIVGKQDRWRIRDGAADTTIDLAKVVSQLVDANGVLKHSIKLEVIGGDGDDVVLANASRIHYDGGAGTNTVSYAALGRQDSITVSADGERFNVRKQLNNANVYREGVATQKTAYGKRTENVQYRHVELARVGQLVEVDTLEHVQHIIGGAGNDSITGNAHDNFLAGGAGDDRLDGGAGNDTLVGGEGHNTVVGGAGDDVFLQDLGVWSNQLDGGAGVDTVKYNVHQPSEERLERMGETGIHADLQKGTVEKWPALNLFSVDHVKNIENLHGSSLNDSIAGDDRDNELWGDDGNDTIHGRGGDDILRGGLGLDTLYGEDGNDIFLQDDETVSDDIDGGAGLDTVDYSAMIHAGKIVAPHEYGFGIEADLSREWVRKASALGVDYYDSVRNVENVIGTSMKDVLIGDAQANTLMGQGGDDTVRGGDGDDLLFGGDGNDMLYGDAGNDTLYGGLGDDTLEGGAGNDWFGQTPAREHDVLRGGAGVDTVDYSQAGAHAGVATGRIGLGILADLGAGRVDKLGEAGSSAYDTVSGIENVVGTELADRITGDAQANVLRGAGGADVLAGGEGDDVLLGGEGDDQLSGDAGRDRLYGEAGDDWFFQDAANAGNLLDGGDGNDTVDFSGPGRGLDAGAKGVFLSLGKGFASLMDEPETSNVLRHIENAVGSVRDDVLIGDAGANVLNGLAGNDVLSGGAGDDVLLGDEGSDLLSGDAGNDDLFGGQGDDTYLFGAGYGHDTIYESGGGHDTIRINAGADQLWFARQGNDLEIRILGTDDALTVHDWYRDADHRVEAIHAANQAIDPAGIEKLVEAMAQYPDPGAAAAAPPAARVPDTLMQSLAVNWR

SEQ ID NO: 3 - protein, 1706 aa, Bordetella parapertussis
MQQSHQAGYANAADRESGIPAAVLDGIKAVAKEKNATLMFRLVNPHSTSLIAEGVATKGLGVHAKSSDWGLQAGYIPVNPNLSKLFGRAPEVIARADNDVNSSLAHGHTAVDLTLSKERLDYLRQAGLVTGMADGVVASNHAGYEQFEFRVKETSDGRYAVQYRRKGGDDFEAVKVIGNAAGIPLTADIDMFAIMPHLSNFRDSARSSVTSGDSVTDYLARTRRAASEATGGLDRERIDLLWKIARAGARSAVGTEARRQFRYDGDMNIGVITDFELEVRNALNRRAHAVGAQDVVQHGTEQNNPFPEADEKIFVVSATGESQMLTRGQLKEYIGQQRGEGYVFYENRAYGVAGKSLFDDGLGAAPGVPGGRSKSSPDVLETVPASPGLRRPSLGAVERQDSGYDSLDGVGSRSFSLGEVSDMAAVEAAELEMTRQVLHAGARQDDAEPGVSGASAHWGQRALQGAQAVAAAQRLVHAIALMTQFGRAGSTNTPQEAASLSAAVFGLGEASSAVAETVSGFFRGSSRWAGGFGVAGGAMALGGGIAAAVGAGMSLTDDAPAGQKAAVGAEIALQLTGGTVELASSIALALAAARGVTSGLQVAGASAGAAAGALAAALSPMEIYGLVQQSHYADQLDKLAQESSAYGYEGDALLAQLYRDKTAAEGAVAGVSAVLSTVGAAVSIAAAASVVGAPVAVVTSLLTGALNGILRGVQQPIIEKLANDYARKIDELGGPQAYFEKNLQARHEQLANSDGLRKMLADLQAGWNASSVIGVQTTEISKSALELAAITGNADNLKSADVFVDRFIQGERVAGQPVVLDVAAGGIDIASRKGERPALTFITPLAAPGEEQRRRTKTGKSEFTTFVEIVGKQDRWRIRDGAADTTIDLAKVVSQLVDANGVLKHSIKLEVIGGDGDDVVLANASRIHYDGGAGTNTVSYAALGRQDSITVSADGERFNVRKQLNNANVYREGVATQKTAYGKRTENVQYRHVELARVGQLVEVDTLEHVQHIIGGAGNDSITGNAHDNFLAGGAGDDRLDGGAGNDTLVGGEGHNTVVGGAGDDVFLQDLGVWSNQLDGGAGVDTVKYNVHQPSEERLERMGDTGIHADLQKGTVEKWPALNLFSVDHVKNIENLHGSSLNDSIAGDDRDNELWGDDGNDTIHGRGGDDILRGGLGLDTLYGEDGNDIFLQDDETVSDDIDGGAGLDTVDYSAMIHAGKIVAPHEYGFGIEADLSEGWVRKAARRGMGYYDSVRSVENVIGTSMKDVLIGDAQANTLMGQGGDDTVRGGDGDDLLFGGDGNDMLYGDAGNDTLYGGLGDDTLEGGAGNDWFGQTPAREHDVLRGGAGVDTVDYSQAGAHAGVATGRIGLGILADLGAGRVDKLGEAGSSAYDTVSGIENVVGTELADRITGDAQANVLRGAGGADVLAGGEGDDVLLGGEGDDQLSGDAGRDRLYGEAGDDWFFQDAANAGNLLDGGDGNDTVDFSGPGRGLDAGAKGVFLSLGKGFASLMDEPETSNVLRHIENAVGSVRDDVLIGDAGANVLNGLAGNDVLSGGAGDDVLLGDEGSDLLSGDAGNDDLFGGQGDDTYLFGAGYGHDTIYESGGGHDTIRINAGADQLWFARQGNDLEIRILGTDDALTVHDWYRDADHRVEAIHAANQAIDPAGIEKLVEAMAQYPDPGAAAAAPPAARVPDTLMQSLAVNWR

SEQ ID NO: 4 - protein, 232 aa, Bordetella bronchiseptica
MVPHHHHHHSRAWRHPQFGGHHHHHHASTIDLGVSLTSQAGGLQGIDLKSMDIQTLMVYVQGRRAELLTAQMQTQAEVVQKANERMAQLNEVLSALSRAKAEFPPNPKPGDTIPGWDNQKVSRIEVPLNDALRAAGLTGMFEARDGQVTAPGGRGTQVVNGTGVMAGSTTYKELESAYTTVKGMLDTASNTQQMDMIRLQAASNKRNEAFEVMTNTEKRRSDLNSSITNNMR

SEQ ID NO: 5 - protein, 249 aa, Bordetella bronchiseptica
MGTVNGLEQLESIINFEKLTEWTSSNVMEERKIKVYLPRARAATSTIDLGVSLTSQAGGLQGIDLKSMDIQTLMVYVQGRRAELLTAQMQTQAEVVQKANERMAQLNEVLSALSRAKAEFPPNPKPGDTIPGWDNQKVSRIEVPLNDALRAAGLTGMFEARDGQVTAPGGRGTQVVNGTGVMAGSTTYKELESAYTTVKGMLDTASNTQQMDMIRLQAASNKRNEAFEVMTNTEKRRSDLNSSITNNMR

SEQ ID NO: 6 - protein, 1590 aa, Bordetella bronchiseptica
MGTVNGLEQLESIINFEKLTEWTSSNVMEERKIKVYLPRARAATSTIDLGVSLTSQAGGLQGIDLKSMDIQTLMVYVQGRRAELLTAQMQTQAEVVQKANERMAQLNEVLSALSRAKAEFPPNPKPGDTIPGWDNQKVSRIEVPLNDALRAAGLTGMFEARDGQVTAPGGRGTQVVNGTGVMAGSTTYKELESAYTTVKGMLDTASNTQQMDMIRLQAASNKRNEAFEVMTNTEKRRSDLNSSITNNMRTGAAPSRSKFSPDVLETVPASPGLRRPSLGAVERQDSGYDSLDGVGSRSFSLGEVSDMAAVEAAELEMTRQVLHAGARQDDAEPGVSGASAHWGQRALQGAQAVAAAQRLVHAIALMTQFGRAGSTNTPQEAASLSAAVFGLGEASSAVAETVSGFFRGSSRWAGGFGVAGGAMALGGGIAAAVGAGMSLTDDAPAGQKAAAGAEIALQLTGGTVELASSIALALAAARGVTSGLQVAGASAGAAAGALAAALSPMEIYGLVQQSHYADQLDKLAQESSAYGYEGDALLAQLYRDKTAAEGAVAGVSAVLSTVGAAVSIAAAASVVGAPVAVVTSLLTGALNGILRGVQQPIIEKLANDYARKIDELGGPQAYFEKNLQARHEQLANSDGLRKMLADLQAGWNASSVIGVQTTEISKSALELAAITGNADNLKSVDVFVDRFVQGERVAGQPVVLDVAAGGIDIASRKGERPALTFITPLAAPGEEQRRRTKTGKSEFTTFVEIVGKQDRWRIRDGAADTTIDLAKVVSQLVDANGVLKHSIKLDVIGGDGDDVVLANASRIHYDGGAGTNTVSYAALGRQDSITVSADGERFNVRKQLNNANVYREGVATQTTAYGKRTENVQYRHVELARVGQVVEVDTLEHVQHIIGGAGNDSITGNAHDNFLAGGSGDDRLDGGAGNDTLVGGEGQNTVIGGAGDDVFLQDLGVWSNQLDGGAGVDTVKYNVHQPSEERLERMGDTGIHADLQKGTVEKWPALNLFSVDHVKNIENLHGSRLNDRIAGDDQDNELWGHDGNDTIRGRGGDDILRGGLGLDTLYGEDGNDIFLQDDETVSDDIDGGAGLDTVDYSAMIHPGRIVAPHEYGFGIEADLSREWVRKASALGVDYYDNVRNVENVIGTSMKDVLIGDAQANTLMGQGGDDTVRGGDGDDLLFGGDGNDMLYGDAGNDTLYGGLGDDTLEGGAGNDWFGQTQAREHDVLRGGDGVDTVDYSQTGAHAGIAAGRIGLGILADLGAGRVDKLGEAGSSAYDTVSGIENVVGTELADRITGDAQANVLRGAGGADVLAGGEGDDVLLGGDGDDQLSGDAGRDRLYGEAGDDWFFQDAANAGNLLDGGDGRDTVDFSGPGRGLDAGAKGVFLSLGKGFASLMDEPETSNVLRNIENAVGSARDDVLIGDAGANVLNGLAGNDVLSGGAGDDVLLGDEGSDLLSGDAGNDDLFGGQGDDTYLFGVGYGHDTIYESGGGHDTIRINAGADQLWFARQGNDLEIRILGTDDALTVHDWYRDADHRVEIIHAANQAVDQAGIEKLVEAMAQYPDPGAAAAAPPAARVPDTLMQSLAVNWR

(19) United States
(12) Patent Application Publication
(10) Pub. No.: US 2018/0133301 A1
(43) Pub. Date: May 17, 2018

(54) IMMUNOGENIC AND VACCINE COMPOSITIONS FOR USE AGAINST BORDETELLA BRONCHISEPTICA INFECTION

(71) Applicants: École National Vétérinaire de Toulouse (E.N.V.T.), Toulouse (FR); Cyanimal IP, Dremil-Lafage (FR)

(72) Inventors: Séverine Boullier, Toulouse (FR); Alain Bousquet-Melou, Toulouse (FR); Renaud Nalin, Dremil-Lafage (FR); Patrick Cornille, Saint-Cannat (FR)

(21) Appl. No.: 15/354,964

(22) Filed: Nov. 17, 2016

(51) Int. Cl. A61K 39/02

(52) U.S. Cl. CPC A61K 39/099; A61K 2039/6037

(57) ABSTRACT

The present invention in certain embodiments generally relates to an immunogenic composition comprising Bordetella sp. Adenylate cyclase toxin (CyaA) for use in treating Bordetella bronchiseptica infections in non-human animals. More particularly, the present disclosure relates to an immunogenic composition comprising Bordetella sp. Adenylate cyclase toxin (CyaA) vector for use in treating Bordetella bronchiseptica infections in non-human animals. The present invention also relates in certain embodiments to an immunogenic composition comprising a Bordetella sp. adenylate cyclase toxin (CyaA) vector carrying at least one antigen of Bordetella bronchiseptica.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 290142_401_SEQUENCE_LISTING_txt. The text file is about 62 KB, was created on Nov. 17, 2016, and is being submitted electronically via EFS-Web.

### FIELD OF THE INVENTION

The present invention relates to the field of immunology, and in particular to the field of immunogenic and vaccine compositions. It relates to such compositions for use against Bordetella bronchiseptica infection and in particular against canine tracheobronchitis, commonly named kennel cough.

### BACKGROUND OF THE INVENTION

Bordetella bronchiseptica (B. bronchiseptica) is a small, Gram-negative, rod-shaped bacterium of the genus Bordetella and closely related to B. pertussis, the obligate human pathogen that causes pertussis (whooping cough).

Humans are not natural carriers of B. bronchiseptica, which typically infects the respiratory tracts of animals and especially mammals animals such as cats, dogs, pigs, cattle, etc.

In veterinary medicine, B. Bronchiseptica alone, but also combined with other pathogens, leads to a range of pathologies in different non-human animal hosts such as:

In swine, B. bronchiseptica and Pasteurella multocida act synergistically to cause atrophic rhinitis, a disease resulting in arrested growth and distortion of the turbinate in the nasal terminus snout.

In cats, B. bronchiseptica causes tracheobronchitis, conjunctivitis, and rhinitis (upper respiratory tract infection—URI), mandibular lymphadenopathy, and pneumonia. However, URI can also be caused by herpesvirus, calicivirus, Mycoplasma or Chlamydia bacteria.

In dogs, B. bronchiseptica causes tracheobronchitis, commonly named kennel cough or bordetellosis, which is a highly contagious respiratory disease typified by inflammation of the trachea and bronchi. The symptoms are a persistent cough, retching, watery nasal discharge and can be in severe cases, pneumonia, inappetence, fever, lethargy and even death. Even if B. bronchiseptica seems to be the major cause of this disease, other pathogens such as canine adenovirus, parainfluenza virus, and mycoplasma can also cause the symptoms alone or in combination.

Bordetella infection spreads through direct contact (licking, nuzzling), through the air (coughing or sneezing), or via contaminated fomites and can occur between animal species such as, for example, dogs and cats.

Vaccination of non-human animals to prevent tracheobronchitis is commonly used but as, and especially by, vaccine against B. bronchiseptica.

The immunologic mechanism of protection provided by vaccination has not been well defined for Bordetella bronchiseptica. It is known that bacterial endotoxins play a role in causing pathology in both the upper respiratory tract as well as in the lung. Neutralization of those endotoxins by locally produced secretory antibody, most likely of the IgA class, as well as systemically produced IgG antibody that can reach the lung via the serum, would be important in reducing pathology caused by the toxins. It is also expected that secretions containing IgA and serum containing primarily IgG (but also some IgA) antibody to Bordetella bronchiseptica would provide protection in the lungs through transudation (Larson et al., Intern J Appl Res Vet Med, Vol. 11, No. 3, 2013).

There are currently three different types of Bordetella bronchiseptica vaccines licensed in the United States for use in dogs: a killed injectable vaccine for subcutaneous use, Bronchicine®CAe (Zoetis), a live attenuated vaccine for intranasal administration, Bronchi-Shield®III (Boehringer Ingelheim Vetmedica), Vanguard® (Zoetis), Nobivac® (Merck Animal Health), and a live attenuated vaccine to be given orally, Bronchi-Shield®ORAL (Boehringer Ingelheim Vetmedica).

A comparative study has been made of protective immunity provided by oral, intranasal and parenteral Canine Bordetella bronchiseptica vaccines by Larson et al. The killed injectable vaccine Bronchicine®CAe was shown to significantly reduce clinical disease signs. The live attenuated intranasally administered vaccine, Bronchi-Shield®III was considered superior in reducing clinical disease compared to the killed injectable vaccine. Live attenuated vaccines have the advantage to induce immunity after a single dose and have been shown to provide protection after only 72 hours. However, intranasal vaccines are more difficult to administer than an injectable product, and are therefore less popular with veterinarians. The oral Bordetella vaccine, Bronchi-Shield®ORAL, provided immunity that was similar to that induced by the intranasal product (Larson et al., Intern J Appl Res Vet Med, Vol. 11, No. 3, 2013).

Hence, in this challenge study, and also in previous vaccine studies where intranasally administered killed vaccines have been compared with systemically administered killed vaccines, protection from clinical disease has been greater with the live vaccines.

A disadvantage with live attenuated vaccines is that their protection is not really long lasting, so a second and possibly a third vaccination with a live attenuated vaccine must be necessary. Furthermore, while live attenuated vaccines induce local immunity, they may cause significant vaccination reaction, in particular in the respiratory tract after spray vaccination.

A general problem with intranasally given live attenuated vaccines is the fact that the vaccine does not stay in the nose, but leaks away through sneezing, leaking and the like. This is an unwanted situation for two reasons: most of the vaccine bacteria become spread into the environment, and an overdose must be given to assure a sufficient level of vaccination. These disadvantages are however accepted in practice since as motivated above intranasal vaccination is currently considered the only way to obtain at least partial protection.

Accordingly, there remains a need for an immunogenic composition, capable of being safely administered, which provides a long-acting immunoprotection against Bordetella bronchiseptica without deleterious side effects of interference.

### SUMMARY OF THE INVENTION

The present invention generally relates in certain embodiments to immunogenic compositions comprising Bordetella sp. Adenylate cyclase toxin (CyaA) for use for treating Bordetella bronchiseptica infections in non-human animals.

More particularly, the invention relates in certain embodiments to immunogenic compositions comprising Bordetella sp. Adenylate cyclase toxin (CyaA) vector for use for treating Bordetella bronchiseptica infections in non-human animals.

The Bordetella sp. adenylate cyclase toxin (CyaA) vector is capable of retaining the binding activity of native CyaA through its N-terminal domain into target cells and translocate into the cytosol of the antigen-presenting cells (APC). Hence, the adenylate cyclase toxin (CyaA) vector is suitable for delivering molecules, in particular Bordetella bronchiseptica antigens and for eliciting a protective immune response.

The present invention also relates in certain embodiments to immunogenic compositions comprising a Bordetella sp. adenylate cyclase toxin (CyaA) vector carrying at least one antigen of Bordetella bronchiseptica.

### BRIEF DESCRIPTION OF THE DRAWINGS

FIGS. 1A and 1B show clinical follow-up of puppies after Bordetella bronchiseptica challenge.

FIG. 1A: Clinical score determined every day for each dog after bacterial infection.

FIG. 1B: Comparison of the number of coughing/day between each group of puppies (control group and vaccine group)

FIG. 2: Specific IFNγ production by PBMC after CyaA stimulation determined by ELISpot in control (n=3) and vaccinated (n=4) group once a week after vaccination and infection. Results are presented as the mean of spot numbers (±SD) per 10⁶ cells for each group. Arrows indicate the times of vaccination and infection.

FIGS. 3A and 3B show humoral response against CyaA.

FIG. 3A: Total serum IgG specific for CyaA determined by ELISA. Results are presented as the mean (±SD) of OD obtained at serum dilutions of 1/20 000 for control (n=5) and vaccinated (n=10) dogs. P<0.01 for each time point between control and vaccinated dogs from D28 until the end of the experiment (D70).

FIG. 3B: IgG1 and IgG2 Abs specific for CyaA detected by ELISA in sera of vaccinated dogs (n=10) at D28, D49 and D70 after the first vaccine injection. Results are presented as the mean (±SD) of OD obtained at serum dilutions of 1/20 000.

FIGS. 4A, 4B, and 4C show immune response against Bsp22.

FIG. 4A: Specific IFNγ production after Bsp22 stimulation by PBMC determined by ELISpot in control (n=3) and vaccinated dogs (n=4) once a week after vaccination and infection. Results are presented as the mean of spot numbers (±SD) per 10⁶ cells for each group. Arrows indicate the times of vaccination and infection.

FIG. 4B: Total serum IgG specific for Bsp 22 determined by ELISA. Results are presented as the mean (±SD) of OD obtained at serum dilutions of 1/20 000 for control (n=5) and vaccinated dogs (n=10).** P<0.01.

FIG. 4C: Serum IgG1 and IgG2 Abs specific for Bsp22 detected by ELISA in sera of vaccinated dogs (n=10) at D63 and D70 after the first vaccine injection. Results are presented as the mean (±SD) of OD obtained at serum dilutions of 1/20 000.

### DETAILED DESCRIPTION

The present invention generally relates in certain embodiments to an immunogenic composition comprising a Bordetella sp. adenylate cyclase toxin (CyaA) vector carrying at least one antigen of Bordetella bronchiseptica.

The term “immunogenic composition” or “vaccine” or its equivalents, relates to any composition comprising a Bordetella sp. adenylate cyclase toxin (CyaA) or a Bordetella sp. adenylate cyclase toxin (CyaA) vector, which can be used to elicit an immune response in a non-human animal. In one embodiment, the immunogenic composition is suitable for eliciting a T-cell immune response, and in a particular embodiment, Th1.

The immunogenic composition of the present invention can in certain embodiments be used to treat a non-human animal susceptible to Bordetella bronchiseptica caused infections and/or diseases by means of administering the immunogenic composition. Advantageously, an immunogenic composition according to certain embodiments induces a response orientated toward Th-1 phenotype, characterized by IFNγ production and IgG2 synthesis.

The Adenylate Cyclase toxin (CyaA) of Bordetella species in particular of Bordetella pertussis or Bordetella bronchiseptica, is a 1706 residue-long protein (177 kDa).

As used herein, “adenylate cyclase toxin” or “CyaA” or “native adenylate cyclase toxin (CyaA)” or “native CyaA” refers to the adenylate cyclase toxin protein from a Bordetella sp., such as the adenylate cyclase toxin protein from Bordetella pertussis (Accession number CPI 05197, SEQ ID NO:1), the adenylate cyclase toxin protein from Bordetella bronchiseptica (Accession number KDS81064, SEQ ID NO:2), the adenylate cyclase toxin protein from Bordetella hinzii (Accession number AAY57201, SEQ ID NO:1), or the adenylate cyclase toxin protein Bordetella parapertussis (Accession number CAB76450, SEQ ID NO:3).

As used herein Bordetella sp. refers to Bordetella species.

In one preferred embodiment, the adenylate cyclase toxin (CyaA) is from Bordetella pertussis (SEQ ID NO: 1).

In another preferred embodiment, the adenylate cyclase toxin (CyaA) is from Bordetella bronchiseptica (SEQ ID NO: 2).

CyaA is a bifunctional protein that consists of an amino-terminal adenylate cyclase (AC) domain of about 400 residues (N-terminal part) and of a Repeats in ToXin (RTX) cytolysin moiety (Hly) of about 1300 residues (C-terminal part). The Hly moiety inserts into cellular membranes and mediates translocation of the enzymatic AC domain into cell cytosol, where this binds calmodulin and catalyzes conversion of ATP to cAMP, thus subverting cellular signaling. The AC domain appears to be a passive passenger that can be replaced by synthetic polypeptides that can also be delivered into cells by the Hly moiety.

The Adenylate cyclase toxin (CyaA) of Bordetella types, in particular of Bordetella pertussis has been described as a recombinant vector able to deliver efficiently polypeptides, such as antigens, into the cytosol of target sentinel cells commonly named Antigen Presenting Cells (APC).

The biology of the adenylate cyclase from Bordetella pertussis, studied at the Institut Pasteur, led to the discovery that this bacterial protein could be engineered into a potent antigen delivery vector as described in EP0637335 and EP17115047.

One of the main unique features of CyaA stems from its unique mechanism of penetrating into eukaryotic cells. CyaA is able to translocate its N-terminal domain directly across the plasma membrane of target cells, from the extracellular side into the cytosol (Guermonprez, P., Ladant, D., Karimova, G., Ullmann, A., and Leclerc, C. Direct delivery of the Bordetella pertussis adenylate cyclase toxin to the MHC class I antigen presentation pathway. J. Immunol. 1999; 162, 1910-1916.; Bauche, C., Chenal, A., Knapp, O., Bodenreider, C., Benz, R., Chaffotte, A., and Ladant, D. Structural and functional characterization of an essential RTX subdomain of Bordetella pertussis adenylate cyclase toxin. J. Biol. Chem. 2006; 281, 16914-16926; Vojtova-Vodolanova, J., Basler, M., Osicka, R., Knapp, O., Maier, E., Cerny, J., Benada, O., Benz, R., and Sebo, P. Oligomerization is involved in pore formation by Bordetella adenylate cyclase toxin. FASEB J. 2009; 23, 2831-2843; Bumba, L., Masin, J., Fiser, R., and Sebo, P. Bordetella adenylate cyclase toxin mobilizes its 2 integrin receptor into lipid rafts to accomplish translocation across target cell membrane in two steps. PLoS Pathog. 2010; 6, e1000901; Karst, J. C., Barker, R., Devi, U., Swann, M. J., Davi, M., Roser, S. J., Ladant, D., and Chenal, A. Identification of a region that assists membrane insertion and translocation of the catalytic domain of Bordetella pertussis CyaA toxin. J. Biol. Chem. 2012; 287, 9200-9212; Subrini, O., Sotomayor-Pe′rez, A. C., Hessel, A., Spiaczka-Karst, J., Selwa, E., Sapay, N., Veneziano, R., Pansieri, J., Chopineau, J., Ladant, D., and Chenal, A. Characterization of a membrane-active peptide from the Bordetella pertussis CyaA toxin. J. Biol. Chem. 2013; 288, 32585-32598; Uribe, K. B., Etxebarria, A., Marti′ n, C., and Ostolaza, H. Calpainmediated processing of adenylate cyclase toxin generates a cytosolic soluble catalytically active N-terminal domain. PLoS One. 2013; 8, e67648; Veneziano, R., Rossi, C., Chenal, A., Devoisselle, J. M., Ladant, D., and Chopineau, J. Bordetella pertussis adenylate cyclase toxin translocation across a tethered lipid bilayer. Proc. Natl. Acad. Sci. U.S.A. 2013; 110, 20473-20478.

The adenylate cyclase was modified by inactivating the enzymatic activity and rendering it permissive to harbor a variety of polypeptide vaccinal sequences or antigens. This was achieved without altering its high affinity to APC and its capability to translocate into the cytosol of the APC (Fayolle et al., In vivo induction of CTL responses by recombinant adenylate cyclase of Bordetella pertussis carrying viral CD8+ T cell epitopes. J Immunol. 1996; 156(12):4697-706).

CyaA recombinant vectors derived from Bordetella sp. species CyaA, in particular of Bordetella pertussis, have been described as recombinant vectors able to deliver polypeptides, such as antigens, into the cytosol of APC and were constructed by recombinant DNA technology, especially as described in WO 93/21324 or WO 02/22169. These CyaA vectors are genetically modified adenylcyclase toxoids carrying modifications, such as point mutations, deletions or insertions, which can be obtained using usual site-directed or random mutagenesis techniques.

Originally, CyaA recombinant vectors are constructed by inserting deliverable polypeptides into the AC domain at selected residues such as 224-225 or 228-229 or 235-236 or 317-318 as described in EP0637335, but also at 107-108 or 132-133 or 137-138 or 232-233 or 235-236 or 317-318 or 319-320 or 335-336 or 336-337 as described in EP1725259.

A new generation of CyaA recombinant vectors are constructed by truncating the AC domain such as between residues 1 to 373 or residues 1 to 372 as described in EP1317282B1, residues 1 to 300 as described in EP1489092 A1, residues 184 to 320 as EP2875130 A1, residues 228 to 320 as described in EP2690172 A1 or residues 224 to 235 as described in EP2351580 A1.

The CyaA Hly domains were demonstrated to bind specifically to the α_{M}β₂ integrin (CD11b/CD18) (WO02/22169) through its CD11b subunit (Osicka R et al., 2015, eLife, 4:e10766) and so exhibit the capacity to target T-cell epitopes of choice into the CD11b(+) subpopulation of dendritic cells. Immunization of mice with a recombinant CyaA bearing appropriate epitopes led to the induction of strong CD8(+) CTL responses, conferring full protection against a lethal viral challenge and an efficient prophylactic and therapeutic antitumor immunity. In WO02/22169, it has been described that fragments of CyaA encompassing residues 373 to 1706 contain the structure essentially required for interaction with the CD11b/CD18 receptor. More specifically, the amino acid sequence extending from residues 1166 to 1281 comprise a determinant for interaction with the CD11b/CD18 receptor, and more particularly that amino sequence extending from residues 1208 to 1243 are described as critical for the interaction of the toxin with CD11b/CD18 in EP1489092 A1 and EP1633776A1.

Some CyaA recombinant vectors are then constructed by combining deletion of AC domain residues such as residues 1 to 300 combined with minor modification of the Hly domain such as acetylation of the Lysine 860 and Lysine 963 residues as illustrated in EP1633776A1 or substitution of the Glutamic acid 570 residue by a glutamine residue and the Lysine 860 by arginine residue as described in EP2233569B1.

Currently the only CyaA recombinant vector used under clinical development is GTL001 (known as ProCervix® in Europe) developed by Genticel as a first-in-class bivalent Human papillomavirus (HPV) therapeutic vaccine candidate in women infected with HPV 16 and/or HPV 18, before the appearance of papillomavirus related cervical lesions or cancer. This vaccine consists of two CyaA proteins carrying E7 antigens of HPV16 and HPV 18 inserted in the CyaA AC domain.

Masin et al., (Negatively charges residues of the segment linking the enzyme and cytolysin moieties restrict the membrane-permeabilizing capacity of adenylate cyclase toxin, 2016 Scientific Reports 6:29137, Nature Publishing Group) describes that a mutation on a specific part of the Hly domain (between residues 400 to 500 of the CyaA) and especially a substitution of amino acid 445, 446 and 448, have positive impact on the pore forming capacity of the CyaA without altering its AC translocation capacity across target cell plasma membrane. Therefore, some CyaA recombinant vectors can be constructed by combining deletion of AC domain residues with mutation on the Hly domain and especially by amino acid substitution of the residues 445 by an Asparagine (D445N), the residue 446 by an Asparagine (D446N) and of the residue 448 by a Glutamine (E448Q). Hence, as used herein, “Adenylate cyclase toxin (CyaA) vector” excludes the native adenylate cyclase toxin. Hence, “Adenylate cyclase toxin (CyaA) vector” means genetically modified adenylate cyclase toxin (CyaA) of Bordetella sp, by at least one mutation and/or at least one deletion and/or at least one insertion in the native sequence of Bordetella sp (SEQ ID No: 1, 2 or 3). These genetically modifications are
In one embodiment, located in the AC domain of CyaA, Preferably genetically modifications are deletion in the AC domain of at least 100 amino acids, 200 amino acids, 300 amino acids, more preferably 360 amino acids to 370 amino acids and even more preferably 373 amino acids in the AC domain.
In another embodiment, located in the Hly domain of CyaA. Preferably genetically modifications are substitutions and especially substitution of amino acid 445, 446 and 448, and even more preferably substitution of the residues 445 by an Asparagine (D445N), the residue 446 by an Asparagine (D446N) and of the residue 448 by a Glutamine (E448Q)

This Adenylate cyclase toxin (CyaA) vector has an amino acid sequence percentage of identity of at least 75% with the amino acid sequence of a native CyaA protein (i.e. CyaA such as SEQ ID NO:1, SEQ ID NO:2 or SEQ ID NO:3) or a fragment thereof, preferably of at least 79%, at least 80%, or at least 85%, still more preferably of at least 90%, at least 95%, at least 96%, at least 97%, at least 98%, or at least 99% identity.

This Adenylate cyclase toxin (CyaA) vector still possesses high affinity to target Antigen Presenting Cells and capability to translocate into the cytosol of the APC and capacity for the delivery of molecules of interest, in particular Bordetella bronchiseptica antigen.

In an embodiment the adenylate cyclase toxin (CyaA) vector is a genetically modified adenylate cyclase toxin (CyaA) of Bordetella pertussis.

In an embodiment, the adenylate cyclase toxin (CyaA) vector is a genetically modified adenylate cyclase toxin (CyaA) of Bordetella bronchiseptica.

In one preferred embodiment, Adenylate cyclase toxin (CyaA) vector comprises or consist of the amino acid sequence of the adenylate cyclase (CyaA) of Bordetella bronchiseptica or Bordetella pertussis wherein a Bordetella bronchiseptica antigen is inserted in the N terminal part and the AC domain is deleted of at least 100 amino acids, 200 amino acids, 300 amino acids, more preferably 360 amino acids, more preferably 370 amino acids and even more preferably 373 amino acids.

The immunogenic composition comprising a Bordetella sp. Adenylate cyclase toxin (CyaA) vector carries at least one antigen of Bordetella bronchiseptica.

The term “antigen” generally refers to a protein, a peptide or a polypeptide, which contains at least one epitope to which a cognate antibody can selectively bind; or that can stimulate the production of antibodies or T-cell responses, or both, in an non-human animal, including compositions that are injected or absorbed into this non-human animal. The immune response may be generated to the whole molecule, or to one or more various portions of the protein, the peptide or the polypeptide (e.g., an epitope or hapten). The term “antigen” includes all related antigenic epitopes. An antigen is recognized by antibodies, T-cell receptors or other elements of specific humoral and/or cellular immunity. The term “antigen” includes all related antigenic epitopes. Furthermore, for purposes of the present disclosure, the antigen can be derived, obtained, or isolated from bacteria, more particularly from Bordetella bronchiseptica. The antigen can consist in a full-length antigen of Bordetella bronchiseptica or an antigenic fragment(s).

Typically, the antigen of Bordetella bronchiseptica is selected from Bsp22, pertactin, filamentous hemagglutinin (FHA), BteA (also named BopC), or any antigen of Bordetella bronchiseptica involved in bacterial colonization or in virulence.

Typically, Bsp22 is a self-associating tip complex protein from the type III secretion system (T3SS) of Bordetella bronchiseptica.

Typically, pertactin is an autotransporter protein of Bordetella bronchiseptica (Inatsuka et al., Pertactin is required for Bordetella species to resist neutrophil-mediated clearance, infection and immunity, July 2010, p. 2901-2909).

Typically, the filamentous hemagglutin (FHA) is an adhesin of Bordetella bronchiseptica (Romero et al., Filamentous hemagglutinin of Bordetella pertussis: a key adhesion with immunomodulatory properties? Future Microbiol. (2014) 9(12), 1339-1360).

Typically, BteA, also named BopC, is a protein secreted from the Bordetella bronchiseptica type III secretion system (Kuwae et al., BteA Secreted from the Bordetella bronchiseptica Type III Secretion System Induces necrosis through an Actin Cytoskeleton Signaling Pathway and Inhibits Phagocytosis by Macrophages, PLOS ONEDOI:10.1371/journal.pone.0148387, February 2016), (Kuwae et al., BopC is a novel type III effector secreted by Bordetella bronchiseptica and has a critical role in type III-dependent necrotic cell death, the journal of biological chemistry vol. 281, NO. 10, pp. 6589-6600, Mar. 10, 2006).

Preferably, the antigen of Bordetella bronchiseptica is the Bsp22 antigen.

As used herein, the term “Bsp22 antigen” includes whole Bsp22 antigen and any Bsp22 antigenic fragments.

In one embodiment, the amino acid sequence of Bsp22 antigen is described in SEQ ID No: 4.

In another embodiment, Bsp22 antigen is conjugated to a linker.

In another preferred embodiment, the amino acid sequence of Bsp22 antigen conjugated to the linker is described in SEQ ID No: 5.

In another embodiment, Bsp22 antigen is any polypeptide having an amino acid identity between 60% and 100%, preferably more than 80% and even more preferably more than 90%, with a part of SEQ ID NO:5 having the same size.

In a most preferred embodiment, the sequence of the Bordetella bronchiseptica adenylate cyclase toxin (CyaA) vector carrying the Bsp22 antigen from Bordetella bronchiseptica is as set forth in SEQ ID NO: 6.

In yet another embodiment, the immunogenic composition further comprises at least one adjuvant.

The term “adjuvant” as used herein refers to a compound or mixture that enhances the immune response when administered together with an immunogen or antigen.

Typically the adjuvant is chosen from among Complete Freund's Adjuvant (CFA), Incomplete Freund's Adjuvant (IFA), montanide ISA (incomplete SEPPIC adjuvant), muramyl peptides such as muramyl dipeptide (MDP) MDP-Lys (L18) (N.sup..alpha.-acetylemuramyl-L-alanyl-D-isoglutaminyl-N.sup.esteoroyl-L-lysine), zinc sulphate, colloidal iron hydroxide, calcium phosphate or calcium chloride, CpG oligodeoxynucleotides (CPG ODN) such as CPG ODN 1826 and CPG ODN 2007, MF59 which is a detergent stabilized oil-in water emulsion containing 5% squalene (w/v), 0.5% Tween® 80 (w/v) and 0.5% Span (w/v) in water, polysaccharides (such as Inulin) and liposomes (such as cationic liposomes, ISCOMs), molecules which have the capacity to activate T-cell immune response, PRP (Pathogen Recognition Receptors), such as adjuvants that bind or are agonists to TLR (Toll like receptor) 3, 4, 7, 8 and/or 9 on immune cells (such as APC).

In a particular embodiment, the adjuvant is a TLR ligand, in particular a TLR ligand selected from the group consisting of TLR ligands of class 3, such as Poly IC, Poly-ICLC, TLR ligands of class 4, such as MPL or GLA, TLR ligands of class 9, such as CpG, and TLR ligands of class 7/8, such as Imiquimod.

In a most preferred embodiment, the adjuvant is Poly-ICLC. Poly-ICLC can be purchased from Oncovir Inc, (WA, US) as Hiltonol™.

Moreover, the immunogenic composition as defined herein may be combined or mixed with at least one immunopotentiator, such as at least another adjuvant, or a surfactant or immunomodulatory substances, such as cytokines or chemokines, or a growth factors such as such as GM-CSF or a combination thereof.

By “combined”, it is meant that the immunogenic composition as defined herein and the immunopotentiator are both put in contact with the host, at the same or different time and/or by the same or different modes of administration, preferably at the same site of contact.

In contrast, “mixed” means that the immunogenic composition as defined herein and the immunopotentiator are in the same formulation when administered.

The amounts and concentrations of adjuvants useful in the context of the present invention can readily be determined by the skilled artisan.

In a particularly preferred embodiment, the immunogenic composition comprises a Bordetella bronchiseptica adenylate cyclase toxin (CyaA) vector carrying a Bsp22 antigen, a Poly-ICLC as adjuvant and optionally some pharmaceutically/veterinary-acceptable carriers.

Immunogenic composition and vaccines encompassed by the present invention can further include one or more pharmaceutically/veterinary-acceptable carriers.

A used herein, “pharmaceutically/veterinary-acceptable” refers to those compounds, materials, excipients, compositions or dosage forms which are, within the scope of sound medical judgment suitable for use in contact with the tissues of subjects, especially veterinary subjects, without excessive toxicity, irritation, allergic response or other problem complications commensurate with a reasonable benefit/risk ratio. Pharmaceutically/veterinary acceptable carriers include but are not limited to solvents, dispersion media, coatings, stabilizing agents, diluents, preservatives, antibacterial and antifungal agents, isotonic agents, adsorption delaying agents, and the like. Diluents can include water, saline, dextrose, ethanol, glycerol and the like. Isotonic agents can include sodium chloride, dextrose, mannitol, sorbitol and lactose, among others known to those skilled in the art. Stabilizers include albumin. Preservatives include merthiolate.

In a preferred embodiment, the immunogenic composition is for use for treating a Bordetella bronchiseptica infection in a non-human animal

The term “treatment” or “method of treating” or its equivalent is not intended as an absolute term and, when applied to, for example, infection refers to a procedure or course of action that is designed to reduce or eliminate or to alleviate the symptoms of an infection. A “treatment” or a “method of treating” infection does not necessarily mean that the infection will, in fact, be eliminated, be reduced, or that the symptoms of infection will, in fact be alleviated. Often, a “treatment” or a “method of treating” infection will be performed even with a low likelihood of success but is nevertheless deemed to induce an overall beneficial course of action. In an embodiment, the term “treatment” also includes the prevention of an infection. Instead, prevention, e.g., of Bordetella bronchiseptica infection, refers to delay on onset, reduced frequency of symptoms, or reduced severity of symptoms associated with the disorder. Prevention therefore refers to a broad range of prophylactic measures that will be understood by those in the art. In some circumstances, the frequency and severity of symptoms is reduced to non-pathological levels. In another embodiment, the term “treatment” also includes a prophylactic treatment of non-human animals. A prophylactic treatment aims at preventing the Bordetella bronchiseptica infection of said non-human animal and/or preventing the apparition, the development and/or the occurrence of a pathological state and/or symptoms in said non-human animal. The prophylactic treatment encompasses vaccination.

Typically, the term “non-human animal” encompasses, and preferably is, a non-human mammal such as for example a canine, a feline, a bovine, an ovine, a porcine, a camelid or an equine.

Preferably, the non-human animal is a canine or a feline.

In a more preferred embodiment, the immunogenic composition is for use for treating canine tracheobronchitis, commonly named kennel cough.

Typically, the immunogenic composition is administered in a non-human animal by different routes: by oral or per oral administration, oronasal administration, parenteral administration, intranasal administration, respiratory administration.

“Oral” or “peroral” administration, as used herein, refers to the introduction of the immunogenic composition of the invention, into the non-human animal body through or by way of the mouth and involves swallowing or transport through the oral mucosa (e.g., sublingual or buccal absorption) or both. Intratracheal is also a means of oral or peroral administration.

“Oronasal” administration, as used herein, refers to the introduction of the immunogenic composition of the invention, into the non-human animal body through or by way of the nose and the mouth, as would occur, for example, by placing one or more droplets in the nose. Oronasal administration involves transport processes associated with oral and intranasal administration.

“Parenteral administration”, as used herein, refers to the introduction of the immunogenic composition of the invention, into the non-human animal body through or by way of a route that does not include the digestive tract. Parenteral administration includes subcutaneous, intramuscular, intraarterial, and intravenous administration. For the purposes of this disclosure, parenteral administration excludes administration routes that primarily involve transport of the substance through mucosal tissue in the mouth, nose, trachea, and lungs.

“Intranasal” administration, as used herein, refers to the introduction of the immunogenic composition of the invention, into the non-human animal body through or by way of the nose, and involves transport of the substance primarily through the nasal mucosa.

“Respiratory” administration, as used herein, refers to the introduction of the immunogenic composition of the invention, into the non-human animal body through or by way of inhalation of a nebulized (atomized) substance. In respiratory administration, the primary transport mechanism involves absorption of the atomized substance through the mucosa in the trachea, bronchi, and lungs and is therefore different than intranasal or peroral administration.

The immunogenic compositions defined herein may be in a solid form (capsule, powder, tablet, pill, suppository, quick release tablet, gastro-resistant tablet, delayed release tablet), a powder form, preferably after lyophilization (lyophilized form or lyophilized powder form) which needs to be reconstituted for example with diluents(s) before injection, or in a liquid form, such as an injectable solution or injectable suspension.

Preferably, the immunogenic composition of the invention is administered to a non-human animal subcutaneously in a liquid form.

In another embodiment, the immunogenic composition of the invention is administered to a non-human animal with another vaccine, selected from vaccines for treating others pathogens in Bordetella bronchiseptica infection such as pasteurella multocida, herpesvirus, calicivirus, mycoplasma, chlamydia, adenovirus and parainfluenza virus alone or in combination.

In another embodiment, the immunogenic composition of the invention can be administered to the non-human animal in one dose or in multiple doses.

“Dose” refers to the immunogenic composition given to a subject. A “first dose” or “priming dose” refers to the dose of such composition given on day 0. A “second dose” or a “third dose” or an “annual dose” refers to an amount of such composition given subsequent to the first dose, which can be but is not required to be the same vaccine or immunogenic composition as the first dose.

A particular regimen that may be adopted is a repeated administration protocol, especially in a protocol which encompasses two rounds or more of administration.

Typically, the immunogenic composition is administered in one dose, followed by subsequent doses.

Preferably, the immunogenic composition is administered in one dose, followed by a second dose.

More preferably, the immunogenic composition is administered in first dose, followed by a second dose, the second dose is given 3 weeks after the first dose.

In another preferred embodiment, the first dose and the second dose are administered subcutaneously.

The present invention also relates in certain embodiments to an immunogenic composition comprising a Bordetella sp. adenylate cyclase toxin (CyaA) for use for treating a Bordetella bronchiseptica caused infection and/or related disease.

In one preferred embodiment, the Bordetella bronchiseptica caused infection and/or related disease is tracheobronchitis, commonly named kennel cough.

Preferably, the adenylate cyclase toxin (CyaA) is from Bordetella bronchiseptica.

The invention in certain embodiments also concerns a method for treating of a non-human animal presenting with a Bordetella bronchiseptica infection or suspected to have a Bordetella bronchiseptica infection comprising (a) the administration of an effective amount of the immunogenic composition of the present disclosure into said non-human animal, possibly as multiple administered doses, and (b) the follow up of the condition of said non-human animal.

The invention also concerns in certain embodiments a method for treating of a non-human animal presenting with a Bordetella bronchiseptica infection or suspected to have a Bordetella bronchiseptica infection comprising (a) the administration of an effective amount of the immunogenic composition of the present disclosure into said non-human animal, possibly as multiple administered doses, and (b) the follow up of the condition of said non-human animal, possibly as multiple administered doses.

An “effective amount” of the presently disclosed immunogenic composition refers to the effective amount sufficient to prevent, reduce, eliminate, control, treat or inhibit the Bordetella bronchiseptica infection and/or the symptoms. The term “controlling” is intended to refer to all processes wherein there may be a slowing, interrupting, arresting, or stopping the progression of the diseases and conditions described herein, but does not necessarily indicate a total elimination of all disease and condition symptoms. The doses used for the administration can be adapted as a function of various parameters, in particular as a function of the mode of administration used, of the relevant pathology, or alternatively of the desired duration of treatment. Naturally, the form of the pharmaceutical composition, the route of administration, the dosage and the regimen naturally depend on the condition to be treated, the severity of the illness, the age, weight, and sex of the subject, etc. The ranges of effective doses provided below are not intended to limit the invention and represent preferred dose ranges. However, the preferred dose can be tailored to the individual subject, as is understood and determinable by one of skill in the art, without undue experimentation.

For example, the amount of active agent present in the immunogenic composition of the present disclosure will vary depending upon the particular active agent used, as well as the intended subject for administration of the composition, and route of administration. In general, the immunogenic composition will comprise from about 0.01% to about 90% by weight active agent, preferably from about 10% to about 60% and more preferably, from about 30% to about 50%, based upon the total weight of the immunogenic composition taken as 100% by weight.

In another aspect, the invention concerns in certain embodiments a method for treating an infection in a non-human animal preferably in a canine and/or a feline, wherein the method comprises administering an effective amount of the immunogenic composition of the present disclosure to a non-human animal, preferably a canine or a feline, in need of such treatment. All the embodiments disclosed above for the immunogenic composition are encompassed in this aspect.

In yet another embodiment, the present invention relates to a method for treating a Bordetella bronchiseptica infection, preferably, a method for treating canine tracheobronchitis infection commonly called kennel cough, wherein the method comprises administering a therapeutically effective amount of the immunogenic composition of the present disclosure to a canine in need of such treatment. All the embodiments disclosed above for the immunogenic composition are encompassed in this aspect.

In another aspect, the present invention relates in certain embodiments to the use of Bordetella sp. preferably Bordetella bronchiseptica adenylate cyclase toxin (CyaA) vector for the treatment of Bordetella bronchiseptica infections and more preferably the canine tracheobronchitis infection commonly called kennel cough.

In a further aspect, the present invention concerns in certain embodiments the use of an immunogenic composition comprising a Bordetella sp. adenylate cyclase toxin (CyaA) vector with at least one Bordetella bronchiseptica antigen for the treatment of Bordetella bronchiseptica infection in a non-human animal. In yet another embodiment, the present invention relates to the use of an immunogenic composition for the treatment of a Bordetella bronchiseptica infection comprising a Bordetella sp. adenylate cyclase toxin (CyaA) vector with a Bordetella bronchiseptica antigen for the treatment of the canine tracheobronchitis infection commonly called kennel cough.

All the embodiments disclosed above for the immunogenic composition are encompassed in this aspect.

In another aspect, the present invention relates in certain embodiments to the use of Bordetella sp. preferably Bordetella bronchiseptica adenylate cyclase toxin (CyaA) vector for the manufacture of a pharmaceutical preparation for the treatment of the canine tracheobronchitis infection commonly called kennel cough.

In a last aspect, the present invention concerns in certain embodiments the use of an immunogenic composition comprising a Bordetella sp. adenylate cyclase toxin (CyaA) vector with at least one Bordetella bronchiseptica antigen for the manufacture of a pharmaceutical and/or vaccinal preparation for the treatment of an Bordetella bronchiseptica infection in a non-human animal All the embodiments disclosed above for the immunogenic composition are encompassed in this aspect.

In a further embodiment, the invention relates to the use of an immunogenic composition for the manufacture of a pharmaceutical preparation for the treatment of a Bordetella bronchiseptica infection, comprising a Bordetella sp. adenylate cyclase toxin (CyaA) vector with at least one Bordetella bronchiseptica antigen for the treatment of the canine tracheobronchitis infection commonly called kennel cough. All the embodiments disclosed above for the immunogenic composition are encompassed in this aspect.

### EXAMPLES

### Example 1: Construction, Production and Purification of CyaA Toxoid

Antigens: The synthetic peptide corresponding to antigens such as Bsp22 of Bordetella Bronchiseptica (SEQ ID No: 4) was purchased from Polypeptide (Strasbourg, France).

Construction of recombinant CyaA toxoid: Recombinant CyaA toxoid comprising the antigens such as Bsp22 (CyaA-Bsp22) genetically linked to the N-terminal part of Bordetella Bronchiseptica CyaA protein (SEQ ID NO:6) were produced in E. coli XL1-Blue (Stratagen, an Agilent Technologies Company, USA) transformed with appropriate pT7CACT1-derived constructs (Osicka R. et al., 2000, Infect. Immun., 68: 247-256). Exponential 500-ml cultures were grown at 37° C. and induced by isopropyl 1-thio-β-D-galactopyranoside (IPTG, 1 mM) for 4 h before the cells were washed with 50 mM Tris-HCl (pH 8.0), 150 mM NaCl, resuspended in 50 mM Tris-HCl (pH 8.0), 0.2 mM CaCl2, and disrupted by sonication. Upon centrifugation at 25,000×g for 20 min, the insoluble cell pellets were resuspended in 8 M urea, 50 mM Tris-HCl (pH 8.0), 50 mM NaCl, 0.2 mM CaCl2. Upon centrifugation at 25,000×g for 20 min, clarified urea extracts were loaded onto a DEAE-Sepharose column equilibrated with 8 M urea, 50 mM Tris-HCl (pH 8.0), 120 mM NaCl. After washing, the CyaA toxoid was eluted with 8 M urea, 50 mM Tris-HCl (pH 8.0), 2 M NaCl, diluted four times with 50 mM Tris-HCl (pH 8.0), 1 M NaCl buffer, and further purified on a phenyl-Sepharose column equilibrated with the same buffer. Unbound proteins were washed out with 50 mM Tris-HCl (pH 8.0), and the CyaA toxoid was eluted with 8 M urea, 50 mM Tris-HCl (pH 8.0), 2 mM EDTA and stored at −20° C. Concentrations of the purified CyaA toxoid were determined by the Bradford assay (Bio-Rad, Hercules, USA) using bovine serum albumin as a standard. All purified CyaA-Bsp22 toxoid was more than 90% pure as judged by SDS-gel analysis.

### Example 2: Materials and Methods for the Evaluation of the Immunogenic Composition

This study was approved by the Ethical Committee of MENSER (number 201509155295755).

### Animals

Beagle dogs were purchased from a licensed kennel for animal breeding and husbandry (CEDS, Centre d'Elevage du Domaine de Souches, France). Specific pathogen-free puppies were eight weeks old at their arrival. They were bred in a specific facility free of Bordetella bronchispetica. Before the beginning of the study, all the puppies were shown to be seronegative for Bordetella bronchiseptica. All dogs were dewormed and vaccinated against canine distemper virus, parvovirus, leptospirosis, infectious canine hepatitis and parainfluenza virus. They were housed in an accredited class II facility and were provided commercial dog food (VetCare Nutrition, Junior 10 kg, Royal Canin) and tap water ad libitum.

In total, 15 dogs were used, 8 females and 7 males. Dogs were randomly allocated to two groups, following an equivalent ratio of male vs female in each group. The control group was composed of 5 puppies and the vaccinated group 10

### Vaccination Preparation, Formulation and Regimens

Dogs were vaccinated with the CyaA-Bsp22 toxoid as prepared in Example 1.

Each vaccinated dog received 2 subcutaneous injections of 300 μg CyaA-Bsp22 toxoid (in Urea 2.4M) and 150 μg of Poly-ICLC (Oncovir) at 3 weeks interval (“vaccine group”). Control dogs received the same amount of adjuvant (Poly-ICLC) in 2.4M Urea (“control group”).

All dogs received two parenteral vaccine injections at 10 weeks of age (2 weeks after their arrival) and three weeks later (DO and D21). The control group received the same amount of excipient and adjuvant as in the vaccine formulation.

All dogs were infected by Bordetella bronchiseptica four weeks after the second vaccine injection (D49). All puppies were exposed to an aerosol of 1 ml of challenge preparation (8 109 CFU/ml) during 15 min using a nebuliser (OMRON, compAir Pro C900). Puppies were slightly tranquilized with an IM injection of a mix of butorphenol (0.3 mg/kg) and acepromazine (0.01 mg/kg) 10 min before the aerosol nebulization.

### Bacterial Strains and Infection Procedure

Bordetella bronchiseptica strains were purchased from the Pasteur Institute Collection (CRBIP4.201 and CRBIP4.202) and ATTC (Bb 31124 Moreno-Lopez). Strains were inoculated on Bordet Gengou agar plates supplemented with 15% defibrinated sheep blood (Biomerieux, France) and incubated aerobically for 48 hours at 37° C. Strains were then transferred to Stainer-Scholte broth and incubated, with shaking, under aerobic conditions for 18 hours at 37° C. The cultures were stopped when the DO at 600 nm reached 1. The bacterial count at D.O.=1 was determined to be 109 cfu/ml.

The challenge preparation was an equal mix of the three bacterial strains, corresponding to a total of 8.0 109 CFU/ml.

### Clinical Follow-Up

Two clinical follow-ups were performed.

A clinical follow-up was performed daily from the day before each vaccine injection and until 7 days post vaccination following the criteria presented in table 1.

TABLE 1
Rectal Temperature (° C.):
Weight (kg):
Lethargy

| | MACROSCOPIC ANALYSES | | | | |
|---|---|---|---|---|---|
| | | | | Erosion/ | Other |
| | Erythema | Induration | Oedema | Ulceration | problems |
| CLINICAL | Absent | Absent | Absent | Absent | |
| SIGNS | Mild | Mild | Mild | Mild | |
| | Severe | Severe | Severe | Severe | |

A clinical follow-up was performed daily from the day before challenge and during one month post infection following the criteria presented in table 2.

TABLE 2
Rectal Temperature (° C.):
Weight (kg):

| | Qualifier | Score |
|---|---|---|
| Lethargy | | |
| Cough | Absente | 0 |
| | Induced | 1 |
| | Spontaneous | 3 |
| Nasal Discharge | Clear | 1 |
| | Thick | 2 |
| Sneezing | Frequent | 1 |
| Vomiting | | 5 |
| Dyspnea | | 10 |

### Isolation of PBMC (Peripheral Blood Mononuclear Cells)

Sampling was performed every week, starting on the day of the first immunization until day 70 after the primo-vaccination. Blood samplings were performed at the jugular vein and blood was collected in heparinized tubes. Seven dogs (3 controls and 4 vaccinated) were chosen at random at their arrival for the follow-up of the cellular immune response.

PBMC were immediately isolated from whole blood by density gradient centrifugation using Histopaque®1077 (density: 1.077 g/mL, Sigma-Aldrich).

After centrifugation and washings, the resulting cell pellet was suspended in a small volume of complete medium and the viable cell concentration was determined by microscopic examination using Gibco® trypan blue exclusion. Viability was >90% in all samples.

### IFNγ ELISpot

The cells were cultured in complete medium composed of Gibco® Roswell Park Memorial Institute (cRPMI) Medium 1640 containing GlutaMAX® (L-alanyl-L-glutamine) and HEPES, supplemented with 1% sodium pyruvate, 1% non-essentials aminoacids, 100 μg/ml streptomycin, 100 IU penicillin and 10% heat-inactivated Gibco® Fetal Bovin Serum (cat 10270, batch 41G7832K) at a concentration of 2×10⁶ PBMC/ml. PBMC were incubated in triplicate in 96 well PVDF plates (Mabtech) previously coated with 0.75 μg/well of equine IFNγ capture antibody (bIFNγ-I) at 37° C., 5% CO2 for 20-24 h.

For each dog and at each time point, cells were cultured in complete medium as a negative control and in the presence of 50 ng/ml Phorbol 12-Myristate 13-Acetate and 1 μM ionomycine calcium salt (Sigma-Aldrich) as a non-antigen specific positive controls to assess the functionality of the PBMC. The antigen-specific cellular response against the vaccine was tested with 0.5 μg/well of CyaA or 0.5 μg/well of Bsp22.

Plates were washed five times with PBS and 0.025 μg specific anti equine IFNγ biotinylated antibody (Mabtech) was incubated 2 hours at room temperature. After five washed, plates were incubated with 1:1000 streptavidin-ALP (Mabtech) and BCIP/NBT chromogen (Mabtech). The number of spots was determined with an automated ELISpot reader (CTL®).

For each point, the geometric mean of spot number was calculated (mean of triplicate). For the specific response against CyaA or Bsp 22, the data are presented as the number of spots per 10⁶ cells after stimulation with CyaA (or Bsp22) minus the value obtained with the negative control obtained with medium alone the same day.

### ELISA (Enzyme-Linked ImmunoSorbent Assay)

Blood samplings were performed at the jugular vein and blood was collected in uncoated tubes in all dogs. Sera were stored at −80° C. until tested.

ELISA testing was performed every week, starting on the day of the first vaccination until day 70 post-vaccination to measure the level of total IgG, IgG1 and IgG2 antibodies specific for CyaA and Bsp 22.

Briefly, the technique was performed as follows:

A 96 well MAXISORP plate (Nunc) was coated with 0.25 μg of purified CyaA or 0.5 μg Bsp 22 per well in carbonate buffer (pH 9.6) overnight at +4° C. Nonspecific sites were blocked with PBT/BSA (PBS+0.1% Tween-20+1% Bovine Serum Albumin) for 2 hours at room temperature. Serial two-fold dilutions of sera were tested starting at 1/5000 until 1/40000. The first dilution that did not saturate the ELISA in vaccinated dogs was used to analyse the results. After 1 hour of incubation at room temperature, plates were washed five times with PBT. A specific peroxidase-conjugated rabbit polyclonal anti-dog IgG secondary Ab (304-035-0030, Jackson Immuno Research) was added at 0.04 μg/well and incubated for 1 hour of incubation at room temperature. After five washes, revelation was done by incubation with TMB Liquid Substrate System (Sigma).

For ELISA detecting dog IgG1 or IgG2, the protocol was similar. A specific peroxidase-conjugated goat polyclonal anti-dog IgG1 secondary Ab (Bethyl Laboratory) was added at 0.02 μg/well and a specific peroxidase-conjugated sheep polyclonal anti-dog IgG2 secondary Ab (Bethyl Lab) was added at 0.002 μg/well.

Plates were read at 450 nm with μQuant™ (Biotek). To standardize the results between plates, a positive dilution range was used on each plate.

### Statistical Analysis

All statistical analyses were performed using the following software: www.socscistatistics.com/tests/mannwhitney/Default2.aspx. The significant threshold was set at p=0.05. Intergroup comparisons of the results were performed using a Mann Witney test.

### Example 3: Safety of the Immunogenic Composition Comprising CyaA Vector Carrying Bsp22 Antigen

In order to evaluate the tolerance of the vaccine, complete general and local clinical examinations were performed the day before and after each injection once a day for 7 days. Examinations were performed according to Table 1.

Immunized dogs did not present alterations of their general condition. No sign of lethargy or appetite loss was identified. No sign of hyperthermia was detected, rectal temperature ranges were between 37.6 and 39.3 for all puppies.

At the injection site, no local reaction was observed, neither after the first injection nor after the second injection.

These results demonstrated that the candidate vaccine was very well tolerated in young puppies.

The immunogenic composition comprising CyaA vector carrying Bsp22 antigen was confirmed to be safe.

### Example 4: Efficacy of the Immunogenic Composition Comprising CyaA Vector Carrying Bsp22 Antigen

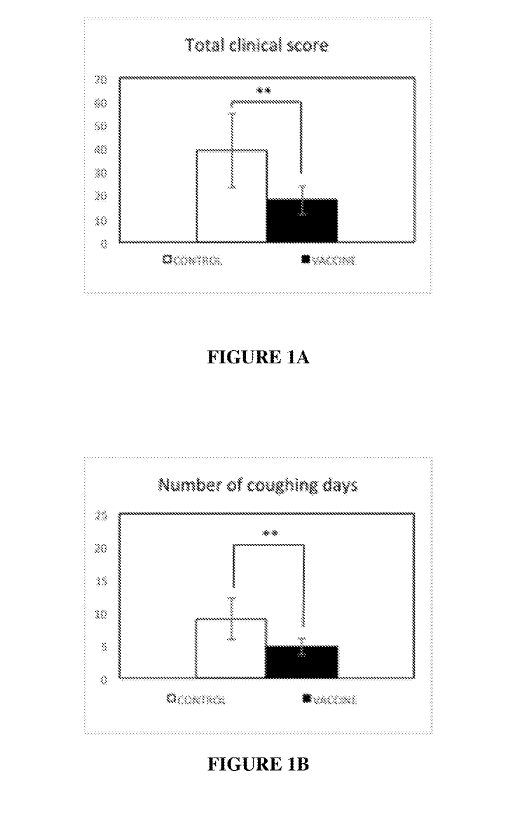

Clinical follow-up of puppies after Bordetella bronchiseptica challenge was performed according to the criteria listed in Table 2 (FIG. 1).

The infection did not induce hyperthermia in both groups of dogs, and no differences in body temperature were observed between vaccinated and control puppies (data not shown).

After infection, control puppies displayed clear clinical signs of tracheobronchitis, including spontaneous and induced coughing, nasal discharge and, for some of them, vomiting. All dogs spontaneously recovered from infection in three weeks.

The mean (±SD) of cumulative score was determined for each group of puppies. P<0.01 between vaccinated (n=10) and control group (n=5) following table 2 criteria (FIG. 1 A). Total clinical score was significantly reduced (p<0.01) in the vaccine group.

For each dog, the number of coughing days was noted (FIG. 1B). Results represent the mean (±SD) for each group of puppies. P<0.01 between vaccinated (n=10) and control group (n=5).

The number of coughing days was significantly reduced (p<0.01) in the vaccine group.

Hence, the immunogenic composition comprising CyaA vector carrying Bsp22 antigen delayed the outbreak of tracheobronchitis and reduced the duration and intensity of cough. The immunogenic composition reduced the intensity and duration of clinical signs after experimental infection.

### Example 5: Immunogenicity of the Immunogenic Composition Comprising CyaA Vector Carrying Bsp22 Antigen

### Cellular Th1 Response

The IFNγ production of PBMC after specific in vitro re-stimulation was quantified by ELISpot, in order to monitor the cellular response induced by CyaA.

The plated cells with Phorbol Myristate Acetate plus ionomycine (PMA+Io) were stimulated to induce a polyclonal T-cell activation (positive control) in order to assess the functionality of our assay. Non-specific activation was detected with cells cultured in the presence of the medium only (negative control).

For each time point and each puppy, an average of 4.3×10³ spots per 10⁶ cells could be detected after PMA+Io stimulation (data not shown), while no spots were detected when cells were cultured in medium alone. These responses were homogeneous among dogs, independently of their status (vaccinated versus placebo) and at the different time points.

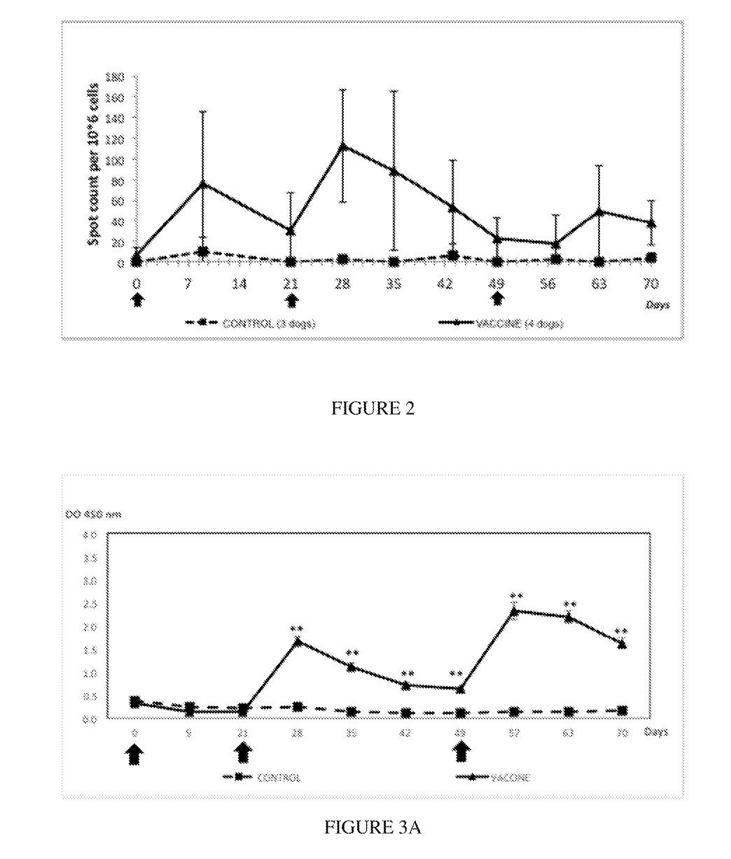

Specific IFNγ production by PBMC after CyaA stimulation was determined by ELISpot in control (n=3) and vaccinated (n=4) dogs once a week after vaccination and infection (FIG. 2). Results are presented as the mean of spot numbers (±SD) per 10⁶ cells for each group. Arrows indicate the times of vaccination and infection.

After in vitro PBMC re-stimulation with CyaA, we observed an early and significant production of IFNγ, as early as 9 days after the first injection between vaccine and control groups (FIG. 2).

This IFNγ production was rapidly boosted by the second injection, with a peak of IFNγ production one week after the second injection, at day 28. At the time of infection (D49), we were still able to detect a cellular response against CyaA in vaccinated puppies. Interestingly, 2 weeks after the infection, the cellular response against CyaA was significantly boosted in vaccinated dog while non-vaccinated puppies did not mount a specific cellular response. Because of the small number of animals in each group, statistical analyses were not possible.

Altogether, our results demonstrate that CyaA induced a specific cellular response skewed toward a Th-1 phenotype in dogs.

### Humoral Response

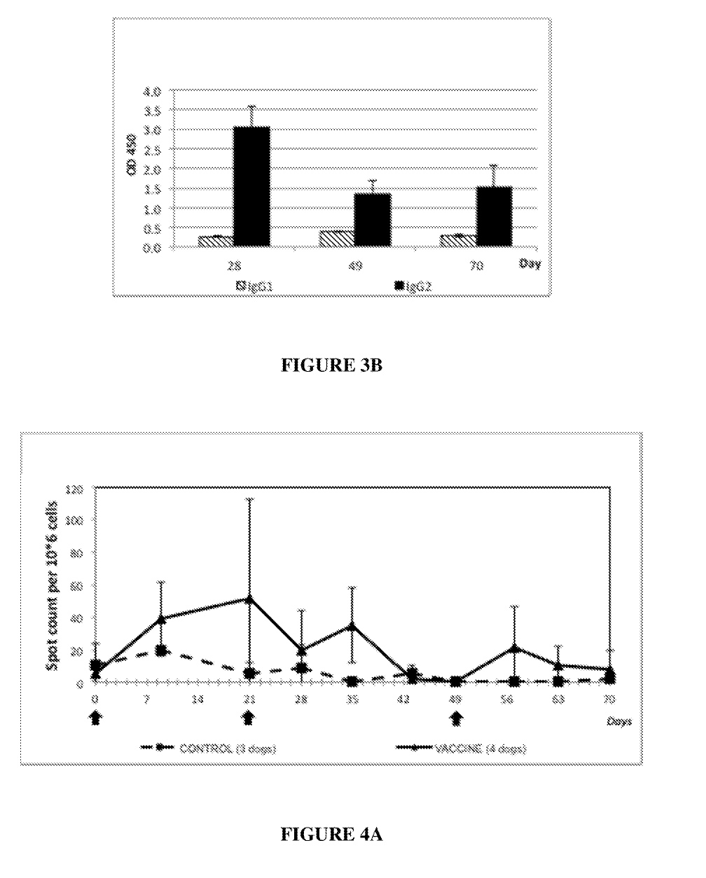

The humoral response induced against CyaA was monitored every week after the first vaccination (FIG. 3).

Over the course of the study, all vaccinated dogs developed a strong humoral response against the CyaA protein (FIG. 3A). This response was detectable one week after the second vaccine injection and was significantly higher than in control dogs during the course of the study (p<0.01). Interestingly, this humoral response was boosted by the virulent challenge in vaccinated puppies while the infection did not induce anti-CyaA antibody synthesis in non-vaccinated puppies.

In order to determine the profile of this humoral response, a CyaA ELISA specific for IgG1 and IgG2 was performed. IgG1 and IgG2 specific for CyaA were detected in vaccinated dogs at the peak of the humoral response one week after the second vaccine injection (D28), just before the infection (D49) and at the end of the experiment (D70) (FIG. 3B). The results clearly indicated that the humoral response was almost exclusively orientated toward the IgG2 subclass, confirming that the vaccine orientated the CyaA response toward a Th-1 phenotype.

### Specific Immune Response Against Bsp22 Antigen

The next step for the validation of the immunogenicity of the candidate vaccine was to demonstrate that it also induced a specific cellular response against the target antigen included in the vaccine i.e. the Bsp22 protein. The IFNγ production was thus quantified by ELISpot by PBMC after in vitro Bsp22 specific re-stimulation.

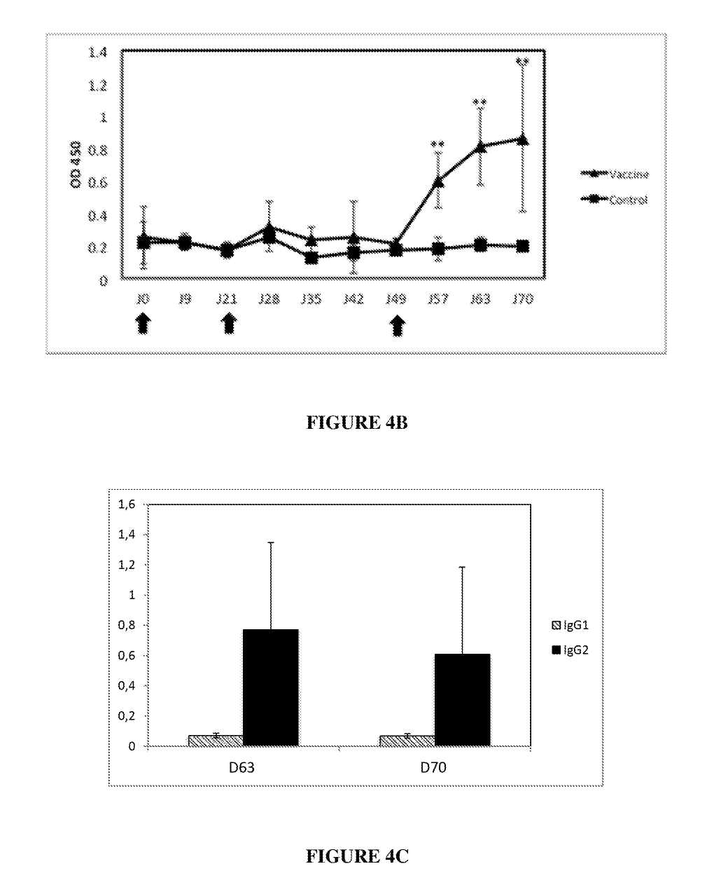

A rapid cellular response detectable was observed as soon as 9 days after the first vaccination (FIG. 4A). Interestingly, the second vaccine did not boost this cellular response. On the opposite, one week after the infection, a boost of the specific cellular response was detectable in vaccinated puppies. No response against Bsp22 was observed in control puppies after the infection. Because of the small number of animals in each group, statistical analyses were not possible.

It has been checked whether a humoral response was also induced against Bsp22 in vaccinated animals. Bsp22-specific IgG Ab were not detectable in vaccinated puppies before the infection (FIG. 4B). However, one week after the infection, we observed a significant and rapid increase in the level of total IgG specific for Bsp22 in the serum of vaccinated puppies (p<0.01). Control dogs did not produce IgG specific for Bsp22 after the infection.

To further qualify this response, the subtype of IgG produced by vaccinated puppies after the infection was determined. IgG1 specific for Bsp22 were not detectable while a strong IgG2 response was observed (FIG. 4C).

These results demonstrate that CyaA efficiently presented Bsp22 antigen to the immune system of puppies. The response induced was orientated toward a Th-1 phenotype, characterized by IFNγ production and IgG2 synthesis. In addition, this response was boosted by the infection.

1. An immunogenic composition comprising a Bordetella sp. adenylate cyclase toxin (CyaA) vector carrying at least one antigen of Bordetella bronchiseptica.

2. The immunogenic composition according to claim 1, wherein the adenylate cyclase toxin (CyaA) vector is selected from a Bordetella bronchiseptica adenylate cyclase toxin (CyaA) vector or a Bordetella pertussis adenylate cyclase toxin (CyaA) vector.

3. The immunogenic composition according to claim 1 wherein the adenylate cyclase toxin (CyaA) vector is a Bordetella bronchiseptica adenylate cyclase toxin (CyaA) vector.

4. The immunogenic composition according to claim 1, wherein the at least one antigen of Bordetella bronchiseptica is selected from Bsp22, pertactin, filamentous hemagglutinin (FHA) and BteA.

5. The immunogenic composition according to claim 4, wherein the antigen of Bordetella bronchiseptica is a Bsp 22 antigen.

6. The immunogenic composition according to claim 5, wherein the Bsp22 antigen is conjugated to a linker.

7. The immunogenic composition according to claim 6, wherein the Bsp22 antigen conjugated to the linker is as set forth in SEQ ID NO: 5.

8. The immunogenic composition according to claim 1, wherein the Bordetella bronchiseptica adenylate cyclase toxin (CyaA) vector carrying Bsp22 antigen of Bordetella bronchiseptica is as set forth in SEQ ID NO:6.

9. The immunogenic composition according to claim 1, further comprising at least one adjuvant.

10. The immunogenic composition according to claim 9, wherein the adjuvant comprises one or more TLR ligands selected from the group consisting of TLR ligands of class 3, TLR ligands of class 4, TLR ligands of class 9, and TLR ligands of class 7/8.

11. The immunogenic composition according to claim 10, wherein the adjuvant is a Poly IC-LC.

12. A method for treating Bordetella bronchiseptica infections and related diseases in a non-human animal comprising administering to said non-human animal an immunogenic composition comprising a Bordetella sp. adenylate cyclase toxin (CyaA) vector carrying at least one antigen of Bordetella bronchiseptica.

13. The method according to claim 12, wherein the non-human animal is a canine or a feline.

14. The method according to claim 12, wherein the Bordetella bronchiseptica infection is canine tracheobronchitis.

15. A method for treating a Bordetella bronchiseptica infection and related diseases in a non-human animal, comprising administering to said non-human animal a Bordetella sp. Adenylate cyclase toxin (CyaA).

16. The method according to claim 15, wherein the adenylate cyclase toxin (CyaA) is a Bordetella bronchiseptica adenylate cyclase toxin (CyaA).

17. The method according to claim 15, wherein the Bordetella bronchiseptica infection is canine tracheobronchitis.
